(19) United States
(12) Patent Application Publication
(10) Pub. No.: US 2022/0338747 A1
(43) Pub. Date: Oct. 27, 2022

(54) SYSTEM AND METHOD FOR MONITORING FLUID MANAGEMENT TO A PATIENT

(71) Applicant: ANTISHOCK TECHNOLOGIES, LTD., Haifa (IL)

(72) Inventors: Yokhai Dan, Regba (IL); Dimitri Khalilov, Tel Aviv (IL); Rami Shinnawi, Haifa (IL)

(21) Appl. No.: 17/623,148

(22) PCT Filed: Jan. 13, 2021
(86) PCT No.: PCT/IB2021/000030

§ 371 Date: Dec. 27, 2021

Related U.S. Application Data
(60) Provisional application No. 62/962,429, filed on Jan. 17, 2020.

(51) Int. Cl. A61B 5/026; A61B 5/00

(52) U.S. Cl. CPC A61B 5/0261; A61B 5/682

(57) ABSTRACT

An optical sensing system includes an optical detector, a rigid support structure for attaching the detector and a coherent light source to a patient and holding those in position, relative to soft tissue, while the coherent light forms a speckle pattern within the tissue in view of the detector. Attaching the detector to the patient via the rigid structure permits hands-free detection of the speckle pattern by the detector. Preferably, the rigid support structure is configured for fitment to teeth of the patient for hands-free positioning of the light source and the detector to image sublingual tissue of the patient and the detector and light source are communicatively coupled to a computer station such that the system is operable for: continuous, hands-free monitoring, by laser speckle imaging, of microcirculation while the patient is in shock, and to manage fluid delivery.

### BACKGROUND

Microcirculation is the circulation of blood through the smallest vessels of the body: arterioles, capillaries, and venules. The primary functions of these vessels are the rapid exchange of gases, water, and metabolites, regulation of blood flow to distal capillaries, and collection of blood from capillaries. The state of a patient's microcirculation may be an indicator of tissue functionality.

Insufficient blood perfusion and oxygen delivery to the tissues resulting from reduced oxygen delivery or increased oxygen consumption leads to a life-threatening condition called shock. Shock is a very common condition in critically ill patients, with several shock forms—such as septic shock, cardiogenic shock, and deep hemorrhagic shock—associated with microcirculation disruptions and failure.

Septic shock, for example, is the most common form of shock. Sepsis is an uncontrolled host response to infection resulting in life-threatening organ dysfunction. Septic shock is an advanced stage of sepsis with an increased mortality rate defined by the need for vasopressor therapy and elevated lactate levels in the blood following proper fluid resuscitation.

Intravenous (IV) fluid administration is one of the most common interventions in medicine worldwide, playing an important role in the treatment of several medical conditions, especially in hemodynamically unstable, critically ill patients. Nowadays, IV fluids are administered in a non-personalized fashion to treat several medical conditions. The decision of IV fluid dosage is mainly guided by close monitoring of macrohemodynamics parameters (such as blood pressure and cardiac output), without taking microcirculation parameters into account.

For example, during aggressive fluid administration to correct the hypoperfusion state in sepsis patients, 2 of each 3 sepsis patients develop a complication called fluid overload with severe clinical implications: increased mortality; increased morbidity due to vital organs injury; increased length of stay in intensive care units; increased hospital readmissions; and increased need for medical interventions, such as dialysis, thoracocentesis, and the use of diuretics, to evacuate the fluid overload. While macrohemodynamics parameters are closely monitored in such patients, a state of loss-of-coherence between macrohemodynamics and microhemodynamics (flow parameters in small blood vessels of end organs) is very common in critically ill patients. In critically ill patients body fluids are managed on the microcirculatory level, for example, in the capillaries, which suggests that applying fluid resuscitation based on microcirculation parameters could provide an insight to optimize IV fluid dosage.

US 2004/0006263 reports a hand-held probe to measure analyte in a mouth.

US 2009/0269716 shows a probe with a wedge that squeezes and slides portions together.

WO 2015/107109 shows an oral clamp with spring-loaded jaws.

US 2016/0220129 reports a hand-held laser speckle imaging device.

### SUMMARY

The disclosure provides methods and devices for non-invasive, continuous and real-time, operator independent and areal repetitive measurement and analysis of blood flow parameters of small blood vessels, microcirculation diameter ranges of from 10 μm to 200 μm, optionally without applying pressure and without disturbing the flow. Devices of the invention provide a mechanical system and electro-optical system adapted to be positioned in contact with or adjacent to soft tissue which constitutes an accessible anatomical window reflecting systemic microcirculation in a reliable way, such as the oral cavity. Systems and methods of the disclosure are useful for hands-free, continuous monitoring microcirculation in a patient. Systems and methods of the disclosure may be used to detect impaired microcirculation, or the de-coherence between micro- and macro-circulation, and reveal to a clinician whether a patient will benefit, or not, from the continued delivery of fluids. Fluid delivery and shock treatment may thus be guided by the continuous, hands-free microcirculation monitoring. Systems of the disclosure are suitable for use in an intensive care unit or other clinical setting and work alongside, and complement, other vital sign monitoring systems to help provide critical information for managing fluid delivery and other therapeutic interventions and mitigating the harms of sepsis, shock, and patient deterioration.

Systems and methods of the disclosure include a sensor device with a support structure to support a sensor unit adapted to be placed in proximity to or in direct contact with the soft tissue, for example, in the sublingual area. The sensor may include positioning and attachment mechanisms that provide the sensor with one or more degrees of freedom with respect to the patient. The sensor may also include fixation mechanisms that fixedly attach a housing component of the sensor to an anchor separately attached to teeth or that lock the housing and optics into position once the position is set. For example, where the housing includes positioning mechanisms, an operator may be free to position optical elements with respect to a first degree of freedom (e.g., along z axis normal to tissue, or along track of teeth parallel to jaw line), a second degree of freedom (e.g., “pitch” and “yaw”, or rotation around orthogonal axes), third, or more degrees of freedom. Once positioned, the clinician may use a fixation mechanism (e.g., may tighten a set screw) to fix the optics in position within the oral cavity. The disclosure also provides methods for optical signal detection, for example, real-time laser speckle interference methods, including the use of coherent energy sources with one or more wavelengths, synchronically operated, to measure blood flow parameters within small blood vessels, including, real-time blood flow rates related parameters and blood flow heterogeneity in soft tissue. Systems and methods of the disclosure use an oral sensor device and an optical sensing system for measuring microcirculation, useful to guide fluid delivery e.g., when a patient is in shock, avoid fluid overload, optimize patient outcomes in an intensive care unit (ICU), operating room (OR), dialysis clinic, heart failure clinic, emergency room (ER), or emergency department (ED).

In one aspect of the present invention, there is provided a novel method and apparatus for non-invasive, continuous, real-time, operator independent and repetitive areas measurement and analysis of blood flow parameters of small blood vessels, microcirculation diameter ranges of from 10 μm to 200 μm. The apparatus comprises a mechanical system and electro-optical system adapted to be positioned in contact with or adjacent to soft tissue which constitutes an accessible anatomical window reflecting systemic microcirculation in a reliable way, such as the oral cavity, eyes, in-ear tissue, or nose mucosa. The apparatus comprises a support structure to support a sensor unit adapted to be placed in proximity to or in direct contact with the soft tissue, for example, in the oral cavity environment, sublingual area, or buccal area. Another aspect of the invention relates to methods of optical signal detection, for example, real-time laser speckle interference methods, including the use of coherent energy sources with one or more sensors, one or more wavelengths, synchronically operated, to measure blood flow parameters within small blood vessels, including, real-time blood flow rates related parameters and blood flow heterogeneity in soft tissue.

It is an object of the invention to assist clinicians in finding the sweet spot between the hypoperfusion state and fluid overload state, thus improving the patient's morbidity and mortality.

Fluid overload in critically ill patients results from continuous IV fluid administration to patients with disrupted microcirculation with decreased fluid responsiveness. Therefore, it is also an object of the invention to assist clinicians in assessing patient fluid responsiveness status by monitoring fluid responsiveness at the microcirculation level, i.e., the location at which fluids are managed in the body.

A laser speckle interference pattern is a physical phenomenon created by the sum of single coherent energy sources, for example, a transmitter with different phases collected by a detector. Those speckles can indicate the dynamics of the reflected object, for example, the dynamics of blood flow in small blood vessels.

The transmitter comprises a coherent energy source with a coherence that can produce a speckle pattern interference. The speckle pattern is produced by illuminating a rough surface with a coherent energy source that produces single coherent sources over the sample with different phases, which physics phenomenon is based upon the Huygens-Fresnel principle. The collected reflected single coherent sources from the sample form a speckle pattern interference.

Speckle size depends on the optical parameters, such as, optical resolution (maximum distance between two points that can be still distinguished as separate points), magnification of lens (ratio between the object and the image), focal length (measure of how strongly the system converges or diverges light), aperture size (the iris through which light travels), and the transmitter source wavelength. The optical parameters that enable acquiring dynamic information from the illuminated surface, should be selected and calculated from the consideration of speckle size, minimum detected object and geometrical parameters. Speckle size should approximately be double the size of the detector's smallest unit, for example, a pixel.

A transmitter spot on the sample should be larger than the desired field of view (FOV) of the system, and with enough power density, for example, source power no more than 0.2 mW/cm2, in continuous wave (CW), so the detector can detect the light with a high signal to noise ratio (SNR). Speckle pattern interference is correlated with the sample's geometry and dynamics, for example, the geometry of the blood vessels and the dynamics of the blood. The detection of speckle dynamics with the detector through the optical system enables sample dynamics analysis.

Speckle dynamics analysis indicates the changes on the sample. When the changes don't occur, the speckle interference will not fluctuate, which indicates a stationary sample. When the changes do occur, for example, moving blood cells in a blood vessel, speckle changes its spatial shape and intensity over time. Those changes are detected as interference fluctuation in spatial temporal domain. From the integration of speckles fluctuation and statistical properties estimation, such as, mean value and standard deviation, sample dynamics, for example, blood flow parameters in blood vessels can be estimated. The ratio between mean value and the standard deviation can be referred as speckle contrast value.

In certain aspects, the invention provides an optical sensing system. The system include an optical detector; a rigid support structure for attaching the detector to a patient and holding in position relative to tissue of a patient; and a light source positioned on the rigid support structure to emit coherent light into the tissue to form a speckle pattern within the tissue in view of the detector. Attaching the detector to the patient via the rigid structure permits hands-free detection of the speckle pattern by the detector. The optical detector may comprises a single sensor (such as a CMOS image sensor or a photodiode) or an array of sensors. The system may include a processing unit on the rigid support structure that forms a digital signal from light received at the detector from the speckle pattern. In preferred embodiments, the system includes an anchor for attaching rigidly to teeth of a patient, and the rigid support structure includes a mounting tab releasably connectable to the anchor.

The light source may include a lens and a focusing module, the focusing module operable to position the lens to focus on the tissue. Using electrical and optical components in a housing that removably couples to an anchor such as a dental anchor provides for hands free operation and quick installation and quick release of a sensor such as an oral monitor of microcirculation by laser speckle interference. In preferred embodiments, a rigid, immobile dental anchor is attached to the teeth, then, a housing of a sensor is attached to the dental anchor. The housing can be quickly attached and detached from the dental anchor and, by implication, the patient. The rigid attachment through the dental anchor provides for a repetitive measurement over the same area of tissue over time. The rigid dental anchor thus provides a spatial reference in the mouth cavity. The rigidity of the dental anchor with attached housing, plus the focusing module (e.g., with auto-focus operations implemented on a chip such as a field-programmable gate array) provide for meaningful, reproducible, and comparable imaging operations via laser speckle interference imaging over time, from the same region of tissue.

In certain embodiments, the system includes a computer system communicatively coupled to the detector to receive a digital signal from the light received at the detector, the computer system comprising at least one processor coupled to memory containing instructions executable by the processor to cause the computer system to reconstruct spatiotemporal data mapping perfusion in a plurality of capillaries located in the tissue.

The computer system may be operable to: receive a digital signal from the light received from the speckle pattern by the detector; and, measure microcirculation in the tissue from the speckle pattern. Measuring microcirculation may include detecting fluctuation in the spatio-temporal domain of the speckle pattern. The computer system provides to a physician information on blood flow in small blood vessels and/or information on a patient's fluid responsiveness. The system may provide guidance and recommendations to decrease and/or increase fluid delivery to thereby manage fluid delivery levels, e.g., to avoid fluid overload or under delivery. The system can present recommendations based on a patient's fluid responsiveness, helping to guide fluid delivery. In a preferred embodiment, the rigid support structure is configured for positionally secure attachment to teeth of the patient for hands-free positioning of the light source and the detector to image sublingual tissue of the patient and the detector and light source are communicatively coupled to a computer station such that the system is operable for: continuous, hands-free monitoring, via the laser speckle pattern, of microcirculation, helping a clinician to guide or manage fluid delivery to treat shock, avoid hypo- or hyper-perfusion, and provide better treatment. Use of the system provides for better treatment, as fluid delivery is optimized to the patient. The rigid support structure may be configured for positioning within a mouth of, and mounting on teeth of, the patient to maintain the position of the detector relative to soft, sublingual tissue in the mouth while the detector forms an image of the speckle pattern. Preferably the rigid support structure maintains position of the detector relative to the soft, sublingual tissue over time while the detector forms multiple co-registered images of speckle patterns.

Preferred embodiments of the system include an anchor attachable to teeth of a patient, in which the rigid support structure is releasably connectable to the anchor and dimensioned for placement in an oral cavity of the patient. Preferably the light source includes a lens and a focusing module whereby the light source is operable to emit the coherent light focused on palatal or sublingual capillaries within the tissue. Connection of the support structure to the anchor maintains positioning of the light source with respect to the oral cavity and the focusing module provides for multiple in-focus laser speckle interference imaging operations over time. The system may include a flexible connection line extending from the rigid structure, the connection line operable to pass one or more of data, power, fluid, or suction to remote instruments, wherein the rigid structure comprises no flexible or moveable part but the connection line and any focusing module.

Preferably the system includes a computer system communicatively coupled to the receiver and operable to: receive a digital signal from the light received from the speckle pattern by the detector; and measure microcirculation in the tissue from the speckle pattern. Measuring microcirculation may include detecting fluctuation in the spatio-temporal domain of the speckle pattern. The computer system may provide to a physician information on blood flow in small blood vessels and a recommendation to decrease fluid delivery to avoid fluid hypoperfusion or hyperperfusion.

Preferably the system includes an anchor attachable to teeth and the rigid support structure to provide hands-free positioning of the light source and the detector, and the system includes a focusing module to image sublingual tissue of the patient and in which the detector and light source are communicatively coupled to a computer station operable for: continuous, hands-free monitoring, via the laser speckle pattern, of microcirculation of the patient. In some embodiments, the rigid support structure is configured for positioning within a mouth of, and mounting on an anchor attached to teeth of, the patient to maintain the position of the detector relative to soft, sublingual tissue in the mouth while the detector forms an image of the speckle pattern. The rigid support structure maintains position of the detector relative to the soft, sublingual tissue over time while the detector forms multiple co-registered images of speckle patterns.

In related aspects, the invention provides a method of monitoring microcirculation. The method includes positioning a sensor on tissue of a patient, wherein the sensor comprises an optical detector, a rigid housing that holds the detector in position relative to the tissue, and a light source positioned on the rigid housing to emit coherent light to form a speckle pattern within the tissue. The method further includes leaving the sensor positioned on the tissue and performing hands-free imaging of the speckle pattern with the detector and analyzing by a computer system images of the speckle pattern to monitor microcirculation in the patient. The positioning step may include attaching the sensor to an anchor anchored to teeth of the patient. The hands-free imaging may include multiple imaging operations over time, wherein each imaging operation includes moving a lens by a focusing module in the sensor to provide focus for that imaging operation. Optionally, a processing unit on the sensor forms a digital signal comprising the images of the speckle pattern and transmits the digital signal to the computer system, wherein the computer system is operable to reconstruct spatiotemporal data mapping perfusion in a plurality of capillaries located in the tissue.

The method may include attaching the sensor via a quick-release attachment mechanism to an anchor within an oral cavity of the patient and leaving the sensor within the oral cavity for performing the hands-free imaging. The anchor may be mounted on maxillary or mandibular teeth of the patient and the imaging may include emitting the coherent light through a focusing module on the sensor into palatal or sublingual capillaries, respectively, within the tissue. Preferably the anchor is mounted in a position that straddles mandibular teeth of the patient with the detector facing soft tissue in the oral cavity, wherein a mechanism of the anchor grips the teeth with sufficient force to maintain the detector in position relative to the soft tissue.

Preferably the rigid structure grips the teeth sufficiently to maintain a position of the detector (or array of detectors) over time to perform multiple hands-free imaging operations of one region of the soft tissue, or of one region per sensor when an array is included.

In preferred embodiments the rigid structure comprises no flexible or moveable part after installing (other than any one or more flexible tubes or wires extending from the rigid structure and from the mouth of the patent, the tubes or wires passing fluid, suction, power, or data to or from the sensor). The sensor may mount fixedly to teeth of the patient to image sublingual soft tissue by hands-free laser speckle interference imaging to provide continuous monitoring of microcirculation in the patient. In certain embodiments, monitoring microcirculation comprises detecting fluctuation in the spatio-temporal domain of the speckle pattern. The method may include providing, by the computer system, a clinician with information on blood flow in small blood vessels and a recommendation aiding the clinician in managing fluid delivery. In certain embodiments, the rigid structure is configured for fitment to teeth of the patient for hands-free positioning of the light source and the receiver, to thereby image sublingual tissue of the patient and the receiver and light source are communicatively coupled to a computer station operable for: continuous, hands-free monitoring, via the laser speckle pattern, microcirculation while the patient is in shock, and to guide fluid delivery to treat shock or avoid fluid overload (hyperperfusion) or underload (hypoperfusion). Preferably, the rigid structure grips teeth of the patient to hold the receiver and light source in position relative to tissue over time so that registration is maintained during formation of multiple laser speckle patterns in sublingual soft tissue. The method may include providing by the computer system to a clinician fluid delivery guidance to mitigate shock or avoid fluid overload.

Other aspects of the disclosure provide a general oral sensing device. The oral sensing device includes a support structure dimensioned to be placed and left within a mouth of a patient and a sensor on a rigid portion of the structure. Part of the rigid portion has a fixed configuration complementary to tooth or bone of the patient whereby the rigid portion maintains the sensor in position to sense a vital function from a region of soft tissue in the mouth. The rigid portion may maintain relative positioning between the sensor and the region of the soft tissue for multiple measurements over time. Preferably once the structure if placed within the mouth, the sensor provides continuous and hands-free sensing of the vital function. The sensor maybe used for any suitable vital function such as blood pressure, pulse, temperature, respiration, oxygen saturation, a blood sugar level, or a microcirculation parameter.

In certain embodiments, the “fixed configuration” is configured to straddle a tooth of the patient. The fixed configuration may be configured to straddle mandibular teeth of the patient with the sensor positioned to sense the vital function from sublingual tissue. The sensor may include a light detector. The sensor may also include a transmitter on the rigid portion to emit coherent light into the region of tissue. The sensor may include a processing unit on the support structure that forms a digital signal of a speckle pattern sensed by the light detector. The sensor may include means (e.g., a wired connection or a wireless antenna) for transmitting the digital signal from the processing unit to a remote computer system. Preferably the processing unit or the remote computer system analyzes the digital signal to report a parameter of microcirculation of the patient.

In some embodiment, the remote computer system is operable to inform a clinician of one or more of impaired microcirculation, de-coherence between the microcirculation and microcirculation, or a risk of fluid overload. In laser-speckle interference embodiments, a processing unit on the support structure forms a digital signal from a speckle pattern sensed by the light detector and sends the digital signal to computer station, whereby the device and the computer station provide continuous, hands-free monitoring of microcirculation. Preferably, except for any connection line or wire extending from the device, the device is fully rigid with no moving parts.

In some embodiments, the sensor includes a transmitter on the rigid portion to emit coherent light into the region of tissue and the sensor includes a light detector operable to image a laser speckle pattern formed by the coherent light in the region of tissue, and the rigid portion maintains the sensor in position over time to form multiple, co-registered laser speckle images of the region of tissue.

In other related aspects, the disclosure provides a method for sensing a vital function. The method includes placing and leaving a sensing device in a mouth of a patient. The sensing device is hands-free, quick-installing, and quick-release. A rigid, immobile fixation unit is attached to the teeth, a sensor unit/housing is attached to the fixation unit—allowing the sensor unit/housing to be quickly attached and detached. The rigid fixation unite provides for a repetitive measurement over same area over time.

The rigid part is actually a spatial reference in the mouth cavity. The device includes an anchor and a support structure carrying a sensor on a rigid portion of the structure, wherein part of the anchor has a fixed configuration complementary to tooth or bone of the patient whereby the support structure connects to the anchor and maintains the sensor in position to sense a vital function from a region of soft tissue in the mouth. The method includes operating the device for hands-free sensing of the vital function. Preferably, the support structure and anchor share a compatible quick-release mechanism. The anchor is attached to the tooth or bone, and left there. The support structure is detachably attached to the anchor for hands-free measurement. Further, the method includes forming, by a processing unit on the support structure, a digital signal representing the vital function, and receiving the digital signal at a computer system that provides a clinician with information about the vital function. The sensor provides continuous and hands-free (and non-invasive) sensing of the vital function. The vital function may be, for example, blood pressure, pulse, temperature, respiration, oxygen saturation, a blood sugar level, or a microcirculation parameter.

In preferred embodiments, the method uses a rigid sensor for continuous, hand-free microcirculation monitoring by laser speckle interference imaging. In the preferred embodiments, the rigid portion maintains relative positioning between the sensor and the region of the soft tissue for multiple measurements over time. Once the structure if placed within the mouth, the sensor provides continuous and hands-free sensing of the vital function (e.g., a microcirculation parameter). The fixed configuration may be configured to straddle a tooth of the patient. Preferably the fixed configuration is configured to straddle mandibular teeth of the patient with the sensor positioned to sense the vital function from sublingual tissue. Optionally: the sensor comprises a light detector on the rigid portion for emitting coherent light into the sublingual tissue and the method includes forming a digital image of a speckle pattern detected in the tissue by the sensor via a processor on the sensing device. In some embodiments, the sensor comprises an array of light detectors. The array of light detectors provides useful imaging even with heterogeneity of the sublingual microcirculation network. The “detection” may be provided by an array of sensors which help in providing faithful imaging of microcirculation regardless of heterogeneity of the sublingual microcirculation network. The method may include analyzing the digital image at a computer system to report a parameter of microcirculation of the patient. Using the rigid sensor for continuous, hand-free microcirculation monitoring by laser speckle interference imaging, the method may include operating the computer system to inform a clinician of one or more of impaired microcirculation, de-coherence between the microcirculation and microcirculation, or a risk of fluid overload.

### BRIEF DESCRIPTION OF THE DRAWINGS

FIG. 1A shows components of an optical sensing system.

FIG. 1B shows the system with a wire-frame view to illustrate a light source.

FIG. 1C shows an adjustment mechanism of the system.

FIG. 2 shows the speckle pattern.

FIG. 3 shows a system.

FIG. 4A is a top view of two sensors placed in an oral cavity.

FIG. 4B is a cross-sectional view of two of the sensors in an oral cavity.

FIG. 5 is a perspective view of the sensor.

FIG. 6 shows optical elements of a sensor placed in an oral cavity.

FIG. 7 is a block diagram of an electro-optical sensor unit system.

FIG. 8A shows raw speckle data taken by imaging a static system.

FIG. 8B shows speckle interference calculated from images of the system in a static state.

FIG. 8C shows speckle interference calculated from the system in a dynamic state.

FIG. 9 illustrates the spatio-temporal domain of the speckle pattern.

FIG. 10 diagrams a method of monitoring microcirculation.

FIG. 11 shows a vital function sensing device for use an oral cavity.

FIG. 12 shows the vital function sensing device positioned within an oral cavity.

FIG. 13 shows steps of a method for sensing a vital function.

FIG. 14A is an image take with visible (green) light illumination in preclinical trials.

FIG. 14B is an image after laser speckle contrast analysis in the preclinical trials.

FIG. 14C is a combined dynamic flow over white field image from the preclinical trials.

### DETAILED DESCRIPTION

An optical sensing system includes an optical detector, a rigid support structure for attaching the detector and a coherent light source to a patient and holding those in position, relative to soft tissue, while the coherent light forms a speckle pattern within the tissue in view of the detector. Attaching the detector to the patient via the rigid structure permits hands-free detection of the speckle pattern by the detector. Preferably, the rigid support structure is configured for attaching to an anchor attached to teeth of the patient for hands-free positioning of the light source and the detector to image sublingual tissue of the patient. The detector and light source may be communicatively coupled to a computer station such that the system is operable for: continuous, hands-free monitoring, by laser speckle imaging, of microcirculation while the patient is in shock, and to guide fluid delivery to treat shock or avoid fluid overload.

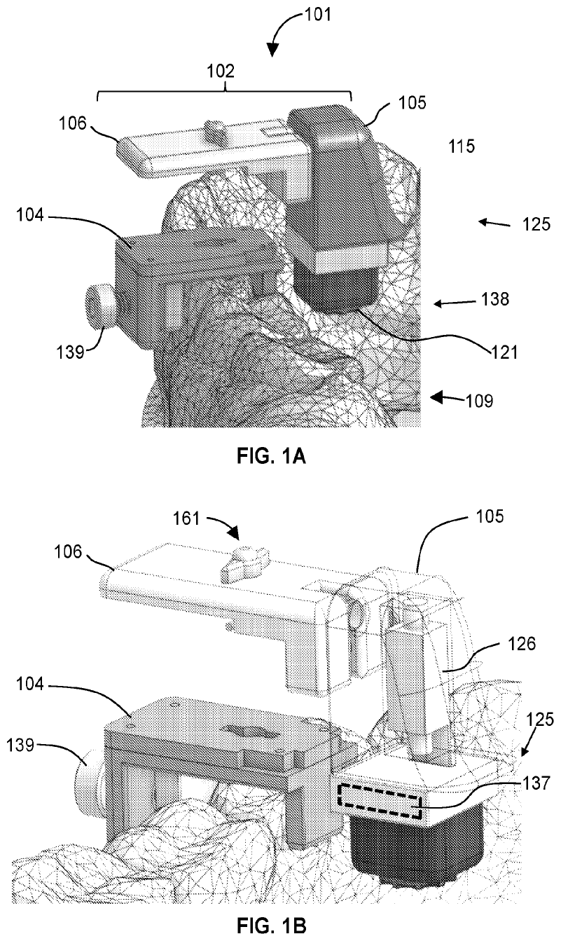

FIG. 1A shows components of an optical sensing system 101. The system 101 includes a sensor 102 that includes an optical detector 121 and a rigid support housing 105 connected to a mounting tab 106 for attaching the detector 121 to an anchor 104. The anchor 104 is configured for attaching to a patient and holding the sensor 121 in position relative to tissue 109 of a patient. The anchor 104 includes a clamp 139 or other suitable mechanism for attaching the anchor 104 to teeth or bone of the patient. Suitable mechanisms may include a press-fit (e.g., a tapered tooth sleeve), cement, a dental screw or other bone screw. In the depicted embodiments, a clamp 139 includes a screw to drive two opposed plates towards each other to securely grip one or more teeth of the patient.

The system 101 includes a light source 125 positioned on the rigid support housing 105 to emit coherent light into the tissue 109 to form a speckle pattern within the tissue 109 in view of the detector 121. Attaching the sensor 102 to the patient via the mounting tab 106 and the anchor 104 permits hands-free detection of the speckle pattern by the detector.

FIG. 1B shows the sensor 102 with the housing 105 drawn in wire-frame view to illustrate placement of optical elements within the sensor 102. A dashed line indicates one location where a processing unit 137 may be included. The housing 105 includes the light source 125 (e.g., a LASER device or semiconductor optical amplifier to provide coherent light)—it is more near 137 (for your judgment if it is ok to change it). The sensor 102 may further include a focusing motor 126 such as a linear piezoelectric motor and associated driver. Any suitable motor may be included such as, for example, an SQL-RV SQUIGGLE motor with NSD-2101 piezo motor driver.

The mounting tab 106 and the anchor 104 share components of an attachment mechanism 161. The attachment mechanism 161 is preferably a quick-release mechanism such as the depicted capstan on the mounting tab 106 and complementary keyway on the anchor 104, but other mechanisms maybe employed such as a lobed cam that clamps to a spindle, or one or more accessible set screws, or keyholes and pegs that create a press-fit.

Preferably, the rigid support housing 105 carries a detector 121 for imaging the soft tissue 109 and the light source 125. The detector 121 and the light source 125 may be mounted to, or connected to, one or more boards (such as printed circuit boards) mounted in the housing 105. The board 603 may also include a processing unit 137 (e.g., such as an FPGA) communicatively coupled to the detector 121 and the light source 125.

In the depicted embodiment, the sensor 102 has a two-part constructions that allows one or more degrees of freedom in positioning the optical elements relative to the anchor 104 as well as provides for fixing the positioning of the optical elements relative to the anchor.

FIG. 1C shows a fixation mechanism 171 of the sensor 102 in the system 101. In the depicted embodiment, the mounting tab 106 is shown fixed (i.e., anchored) to the anchor 104. When the fixation mechanism 171 is disengaged, the housing 105 is free to move relative to the mounting tab 104. For example, where the housing includes positioning mechanisms, an operator may be free to position optical elements with respect to a first degree of freedom (e.g., along z axis normal to tissue, or along track of teeth parallel to jaw line), a second degree of freedom (e.g., “pitch” and “yaw”, or rotation around orthogonal axes), third, or more degrees of freedom. Once positioned, the clinician may use a fixation mechanism (e.g., may tighten a set screw) to fix the optics in position within the oral cavity. Any suitable mechanism may be used such as one or more hinges, or a continuous plastic or elastic material extending to include both the mounting tab 106 and the housing 105. In the depicted embodiment, the mechanism is a ball-and-socket connection (with a ball protruding from the housing corresponding to, and held within, a complementary socket within a notch in the mounting tab 106). The fixation mechanism 171 preferably includes a set-screw to hold the sensor in an engaged position. The depicted embodiment provides for three degrees of movement when the fixation mechanism is in the disengaged position. to maintain a rigid (e.g., no moving parts) spatial relationship between the detector 121 and the tissue 109. The structure 105 preferably includes a processing unit 137 on the rigid support structure that forms a digital signal from light received at the detector from the speckle pattern.

In the depicted embodiment, a light source 125 emits light onto the soft tissue 109 to form a speckle pattern there.

In certain embodiments, the sensor 102 is dimensioned to be positioned and left within an oral cavity 138 of a patient. At least a portion 106 of the sensor 102 may be included to maintain and a fixed and rigid positioning of at least the detector 121 and the soft tissue 109, e.g., by a fitment among the portion 106 of the rigid support structure 105, a skeletal feature of the patient (e.g., teeth or bone), the detector 121, the soft tissue 109, and preferably the light source 125. Once the sensor 102 is positioned and left within the patient, those elements (the rigid support structure 105, a skeletal feature of the patient (e.g., teeth or bone), the detector 121, the soft tissue 109, and the light source 125) do not move relative to one another because of the fitment provided by the rigid support structure 105 and that positioning is maintained over time during sampling time to permit hands-free imaging of the soft tissue 109 to create multiple images that are co-registered to one another (meaning that the multiple images depict or include the same anatomical features of microcirculation) well enough that those images can be superimposed or compared digitally to detect changes over time, or lack thereof, attributable to microcirculation of the patient.

Connections may be entirely wireless. The system may include a flexible connection line 115 extending from the rigid structure 105. The connection line 115 may include any one or more of wire(s) operable to pass data or power or catheter(s) operable to pass fluid or suction fluid, or suction to remote instruments. In most preferred embodiments, other than the flexible connection line 115, the sensor 102 has no flexible or moveable parts.

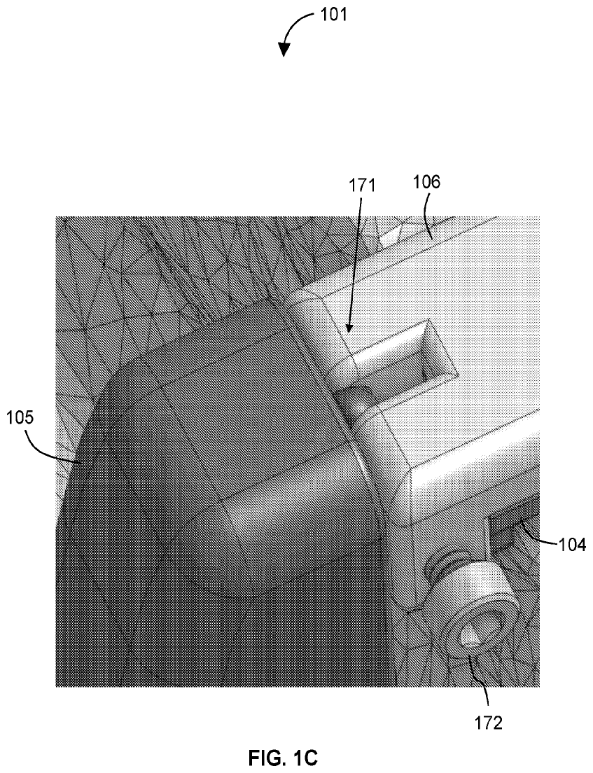

FIG. 2 shows the speckle pattern 201 formed when coherent light is emitted into soft tissue. The system 101 preferably includes means for transmitting a digital signal from the processing unit to a computer system.

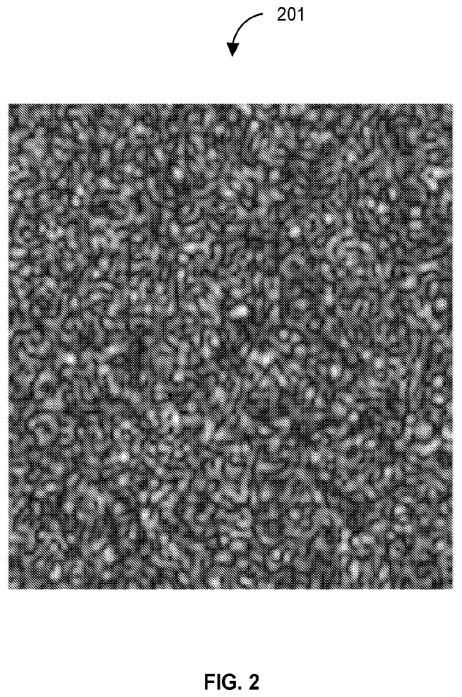

FIG. 3 shows the system 101. As shown, the system 101 includes the support structure 105, which may include a processing unit 137. The system 101 also may include a computer system 301 communicatively coupled to the detector 121 to receive a digital signal 305 from the light received at the detector 121. The system 101 preferably includes means 302 for transmitting the digital signal 305 from the processing unit 137 to the computer system 301. Any suitable hardware device(s) may be used for the means 302 for digital signal transmission. For example, the means 302 may include a wired connection or a wireless antennae, e.g., a data wire extending from the processing unit 137 to part of the computer system 301, or a Bluetooth, near-field connection, network interface card, or other hardware implementing a wireless data transfer protocol. The computer system 301 preferably includes at least one processor coupled to memory (e.g., a tangible, non-transitory computer readable medium; or memory subsystem including local or remote (e.g., server or cloud) data storage) containing instructions executable by the processor to cause the computer system 301 to reconstruct spatiotemporal data mapping perfusion in a plurality of capillaries located in the tissue 109.

Systems and methods of this disclosure provide a reliable continuous and real-time measurement and analysis of blood flow parameters, for example, flow rate in the capillaries, microvascular flow index (MFI), proportion of perfused small vessels (PPV), and inverse decorrelation time (ICT). The system is adapted to measure those parameters in a small noninvasive anatomical window, for example, the oral cavity area, such as a sublingual or buccal area. Measured parameters can include a signal of microcirculatory blood flow in an anatomical window which reflects microcirculatory blood flow in vital organs, thus serving as an anatomical window allowing the evaluation of the systemic circulation in a non-invasive manner.

Systems and methods of this disclosure may provide a clinical decision support closed loop system helping the physician to determine the optimal amount of administered fluids in a personalized fashion. In closed loop embodiments, a fluid delivery system (e.g., an infusion pump) is communicatively coupled to the computer system and the sensors. The computer system measures one or more parameter(s) of microcirculation and relays operational commands to the fluid delivery system. The fluid delivery system response by changing dosage of fluids. The computer system follows up to the change by re-sampling the microcirculation parameter(s). A feedback loop may be implemented whereby the fluid delivery system adjusts delivery in response to commands from the computer system and the computer system continues to re-sample in response to adjusted delivery by the fluid delivery system and update operational commands based on the re-sampling. In a truly closed-loop system, the system manages fluid delivery continuously over a period of time without any intervention by a clinician.

Some embodiments provide a clinician-controlled system (“open loop”), whereby the computer system calculates one or more microcirculation parameters and displays information to the clinician relevant to managing fluid delivery. The clinical operates the fluid delivery system to change fluid delivery according to his or her adjustment, and uses the sensor(s) and computer system to monitor resultant changes in the patient's microcirculation, including evidence of decoherence between micro- and macro-circulation, and/or evidence of hypoperfusion or hyperperfusion.

The data may be inferred from the microcirculation parameters and current fluid administration rate of the patient, the data may be provided to a physician or clinician, and, according to the physician's decision, the fluid dosage may be modified adapted by sending a signal to the fluid management system, for example, an infusion pump system, and all over again. Systems and methods of this disclosure provide measurements that may be delivered to various healthcare clinical decision support and record systems, such as electronic medical record (EMR) and picture archiving and communication system (PACS), reporting tools and patient portals, that can provide an easy access to measured parameters through an easy application programming interface (API).

Systems and methods of this disclosure provide for non-invasive, continuous and real-time measurement and analysis of blood flow parameters in small blood vessels—with a diameter ranging from 10 μm to 200 μm, called the microcirculation. Preferred devices includes one or more electro-optical sensors units with a sensor support unit for continuous and reliable measurement adapted to the anatomical window, for example, the oral cavity 138, environment that works in combination with a side computing device and display to provide physiological measurement parameters for a physician, including trend derivative values, for example, oral microcirculation parameters, such as, sublingual or buccal microcirculation parameters, of a patient. This data generated assists the physician in determining the correct amount of fluid to be administered for each patient according to patient fluid responsiveness status.

The invention includes an apparatus comprising one or more systems, for example, a mechanical system and electro-optical sensor unit system, which is designed according to the geometrical dimensions and physiological conditions of the anatomical window, for example, the human oral cavity.

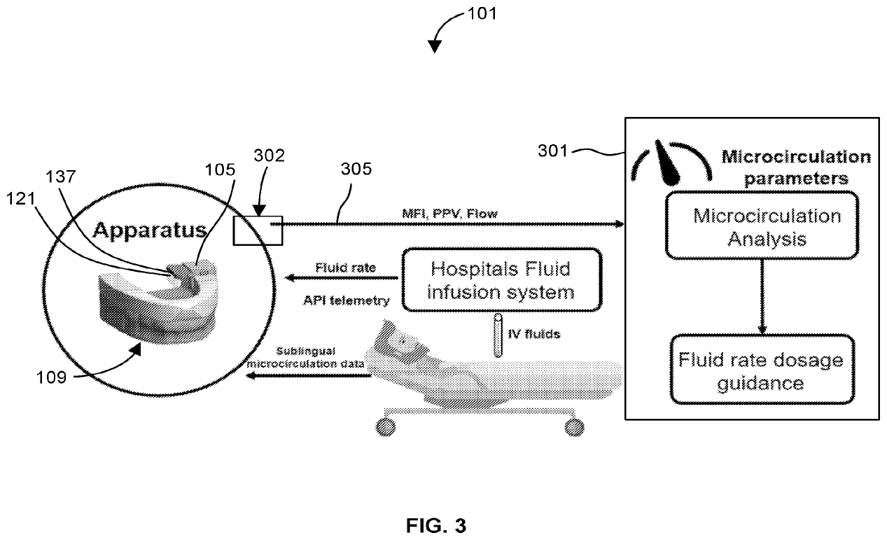

FIG. 4A is a top view of two sensors 402 placed in an oral cavity 138 of a patient according to certain embodiments. As shown, each sensor 02 includes a rigid support housing 405, a mount point 406, and an anchor 404 (anchor visible in FIG. 4B), all dimensioned for placement in an oral cavity 138 of the patient. A dashed line shows an axis of symmetry in the mouth. The figure illustrates an important principle. The sensor 402 may be dimensioned or configured such that a plurality of the sensors 402 may be placed, left, and operated within an oral cavity 138 without interfering with one another. In fact, as shown, the sensor 402 may be dimensioned or configured such that a plurality of the sensors 102 may be placed, left, and operated within an oral cavity 138 without making contact with one another. Optionally, the sensors 402 include a moveable joint 472, such as a ball-and-socket joint, a hinge, or malleable material, as well as a fixation mechanism 471 such as a set screw to lock the housing 405 with respect to the anchor 404.

FIG. 4B is a side view of two of the sensors 402 placed in an oral cavity 138 of a patient. As shown, the rigid support housing 405 is connected to a mount tab 406 attached to an anchor 404 that maintains contact with, and relative positioning of at least the detector 421 and the soft tissue 109 when any attachment or positioning mechanism e.g., joint 472 is engaged and locked e.g., by fixation mechanism 471. Specifically, the anchor 404 connects to one or more teeth 401 in the oral cavity. The mount tab 406 connects to the anchor 404. The housing 405 extends from the mount tab 406. Once installed and fixed in position, repetitive imaging of the same region of tissue 109 over time is provided for by the positioning among the mount tab 406 of the rigid support housing 405, one or more teeth 401, the detector 421, the soft tissue 109, and the light source 425. Once the sensor 402 is positioned and left within the patient, those elements (the rigid support housing 405, the teeth 401, the detector 421, and the light source 425) do not move relative to one another because of the fitting provided by the sensor 402. The optical components such as lenses of the detector 421 or light source 425 may move relative to the tissue 109 under control of a focusing module provided by motors with integrated controller or by focusing logic provided within processing unit 437.

Each sensor 402 is designed to be compact and include a processing unit 437 and means 432 for sending digital signal(s) to a remote computer system. The signal sending means 432 may be a wire connection point (aka a jack or port) or a wireless connection such as a Wi-Fi card or antenna. Exemplified in greater detail below, the sensor 402 includes an optical detector 421, a rigid support structure 405 linked to a mount tab 406 that can be attached to an anchor 404 for attaching the detector 421 to a patient and holding in position relative to tissue 109 of a patient.

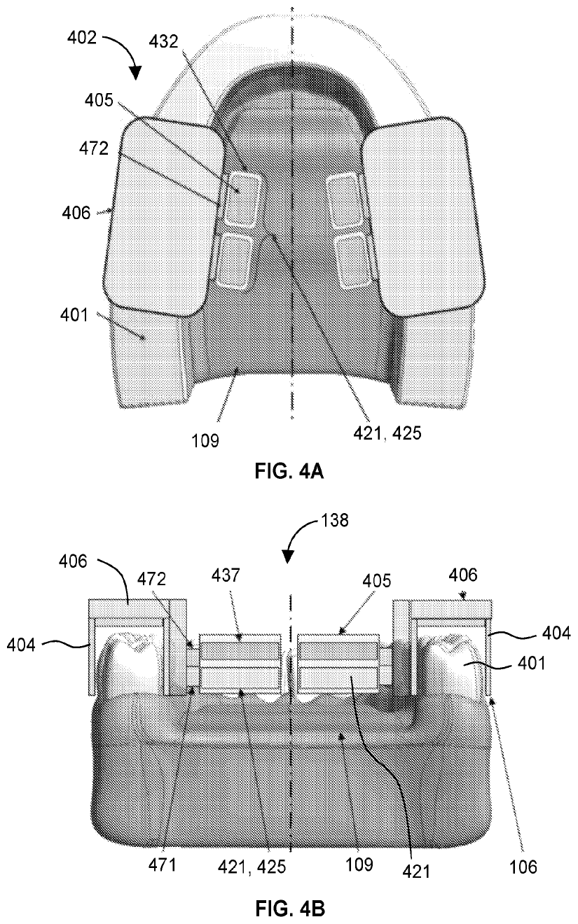

FIG. 5 is a perspective view of a sensor 402. The sensor 402 includes a light source 425 positioned on the rigid support housing 405 to emit coherent light into the tissue 109 to form a speckle pattern within the tissue 109 in view of the detector 421. Attaching the detector 421 to the patient via the sensor 402 permits hands-free detection of the speckle 201 pattern by the detector 121. Importantly, when the rigid housing 405 is fixed in position to the mount tab 406, and the mount tab 406 is attached to the anchor 404, the sensor 402 maintains a rigid (e.g., no moving parts) spatial relationship between the housing 405 and the tissue 109. The only motion is provided by any focusing module in the housing (e.g., a motor and/or focusing module in the light source 425). The housing 405 preferably includes a processing unit 437 that forms a digital signal from light received at the detector from the speckle pattern 201. Shown in greater detail below, a light source emits light onto the soft tissue 109 to form a speckle pattern 201 there.

As shown, the sensor 402 is configured to mount on and straddle mandibular teeth 401 of the patient with a shape and hardware of the anchor 404 plus attachment mechanisms and fixation device providing grip sufficient to maintain the detector 421 in position relative to the soft tissue 109 of the patient.

Thus the disclosure provides an apparatus that includes a mechanical system which comprises one or more units, for example, a mechanical fixation unit, one or more sensor support units, and one or more electro-optical sensor unit subsystems. The mechanical fixation unit includes one or more parts adapted to fixed to the mounting point in the anatomical window, for example, human oral cavity environment tissue, for example, to be mounted to teeth 401 on one or two sides of the lower jaw or/and attached to the buccal surface. The mechanical fixation includes a connection layer which is designed to protect on the mounting point in the anatomical window environment. The mechanical fixation method enables a small range of spatial movement, for example, up to 3 μm in each direction at the measurement time, of the sensor support unit. In addition, there may be a small drift to enable recurrent area detection over time. The small spatial movement enables a stable and reliable and repetitive measurement of microcirculation parameters from repetitive optical signal acquisition of blood flow in multiple capillaries network over time, for example, from a few minutes up to several days, from a selected area.

The sensor support unit includes an electrical interface and mechanical interface. The electrical interface may include a portal through which preferably an input signal initially processed by internal sensor hardware unit 137 and forwarded to postprocessing system 301, which finally converts the response signal into a trend of microcirculatory parameters in the anatomical window, for example, human oral cavity, such as sublingual or buccal area. The mechanical interface provides a rigid support and mechanical focusing to the electro-optical sensor. The focusing mechanism enables a spatial distance calibration of the sensor to the measurement range and preventing a contact interference with the surface. The rigid support provides a fixed location of the electro-optical sensor inside the sensor support unit, which enables acquisition of repetitive measurements from the selected area over time.

The sensor 402 includes a mechanical system and electrical and optical components 601. The electrical and optical components 601 include sensor electrical and opto-mechanical components, such as, optical detector 421, transmitter unit, power supply and management processing unit 437, which may include, for example, a microprocessor or a hardware logical management processing unit, for example, a Field-Programmable Gate Array (FPGA), for managing and signal preprocessing. Furthermore, the electro-optical subsystem includes means 415 for interfaces and protocol conversion, for example, mobile industrial processor interface-camera serial interface (MIPI-CSI), to the postprocessing unit interface, for example, USB or Ethernet.

The electro-optical system is designed and integrated to work synchronically with the postprocessing unit. The electro-optical sensor hardware and mechanics are designed to interface power supply and data transfer to the processing unit. In addition, mechanics may be designed to dissipate heat from the sensor electronics without damaging patient's tissue.

Furthermore, electro-optical system may comprise optical components, for example, a detector lens system and a transmitter coupled lens to expand the transmitter beam that illuminates the microcirculatory anatomical window. As discussed below, the electrical and optical components 601 may include one or more filters, focusing mechanism(s), others, or any combination thereof.

Devices and methods of the disclosure provide for continuous acquisition from an optical signal via one or more electro-optical sensors based on real-time detection technology, for example, a laser speckle interference detector. Laser speckle patterns are produced by illuminating the area, for example, range of 0.5 mm×0.5 mm to 5 mm×5 mm, of capillary network, with one or more coherent light sources, for example, a laser transmitter, and corresponding lenses for each source. The speckle pattern is sensitive to object movement that is converted to fluctuating speckle patterns, in an optical apparatus detector.

It is an object of the invention to assist clinicians in finding the sweet spot between the hypoperfusion state and fluid overload (hyperperfusion) state, thus improving the patient's morbidity and mortality.

Fluid overload in critically ill patients results from continuous IV fluid administration to patients with disrupted microcirculation with decreased fluid responsiveness. Therefore, it is also an object of the invention to assist clinicians in assessing patient fluid responsiveness status by monitoring fluid responsiveness at the microcirculation level, i.e., the location at which fluids are managed in the body.

A laser speckle interference pattern is a physical phenomenon created by the sum of single coherent energy sources, for example, a transmitter with different phases collected by a detector. Those speckles can indicate the dynamics of the reflected object, for example, the dynamics of blood flow in small blood vessels.

The transmitter comprises a coherent energy source with a coherence that can produce a speckle pattern interference. The speckle pattern is produced by illuminating a rough surface with a coherent energy source that produces single coherent sources over the sample with different phases, which physics phenomenon is based upon the Huygens-Fresnel principle. The collected reflected single coherent sources from the sample form a speckle pattern interference.

Speckle size depends on the optical parameters, such as, optical resolution (maximum distance between two points that can be still distinguished as separate points), magnification of lens (ratio between the object and the image), focal length (measure of how strongly the system converges or diverges light), aperture size (the iris through which light travels), and the transmitter source wavelength. The optical parameters that enable acquiring dynamic information from the illuminated surface, should be selected and calculated from the consideration of speckle size, minimum detected object and geometrical parameters. Speckle size should approximately be double the size of the detector's smallest unit—for example, a pixel.

Speckle diameter may be calculated according to Equation 1.

D(speckle)=1.22*(1+M)*λ*f/d (1)

where M is magnification, λ is wavelength (in nm), f is focal length (in mm), and d is the diameter of the lens aperture (in mm).

A transmitter spot on the sample should be larger than the desired field of view (FOV) of the system, and with enough power density, for example, source power no more than 0.2 mWcm2/, in continuous wave (CW), so the detector can detect the light with a high signal to noise ratio (SNR).

Speckle pattern interference is correlated with the sample's geometry and dynamics, for example, the geometry of the blood vessels and the dynamics of the blood. The detection of speckle dynamics with the detector through the optical system enables sample dynamics analysis.

Speckle interference in stationary sample was shown in FIG. 2.

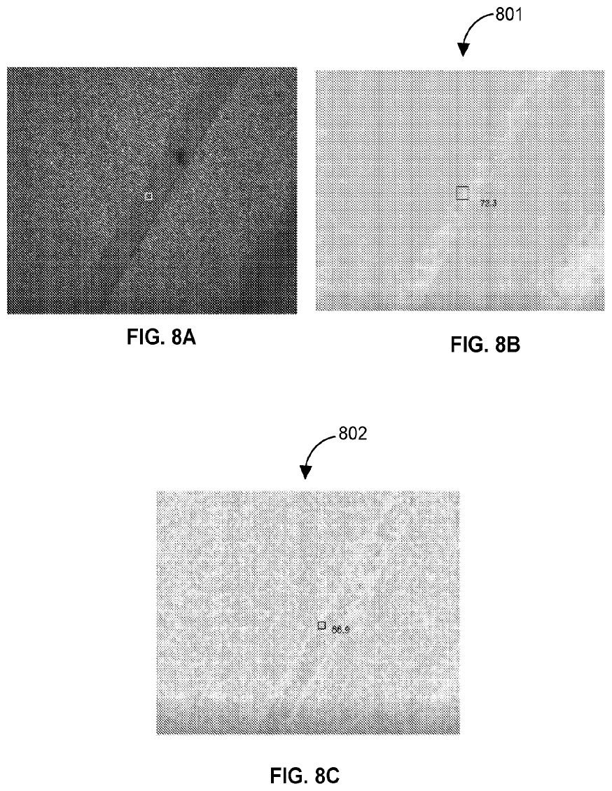

Speckle dynamics analysis indicates the changes on the sample. When the changes don't occur, the speckle interference will not fluctuate, which indicates a stationary sample (FIG. 2). When the changes do occur, for example, moving blood cells in a blood vessel, speckle changes its spatial shape and intensity over time. Those changes are detected as interference fluctuation in spatial temporal domain (FIG. 9).

Any suitable method may be used to calculate speckle contrast. For example, contrast may be calculated using the integration of speckles fluctuation and statistical properties estimation, such as, mean value and standard deviation, sample dynamics, for example, blood flow parameters in blood vessels. The ratio between mean value and the standard deviation can be referred as speckle contrast value. Speckle contrast value K can be calculated according to Equations 2.1 and 2.2.

$$K = \frac{\sqrt{< {I}^{2} > - < I {>}^{2}}}{< I >} \qquad ( 2.1 )$$

$$K = \frac{\sqrt{\frac{1}{{( n + 1 )}^{2}} {\sum }_{x = i - \frac{n}{2}}^{i + \frac{n}{2}} {\sum }_{y = j - \frac{n}{2}}^{j + \frac{n}{2}} {I}_{x , y}^{2} - {( \frac{1}{{( n + 1 )}^{2}} {\sum }_{x = i - \frac{n}{2}}^{i + \frac{n}{2}} {\sum }_{y = j - \frac{n}{2}}^{j + \frac{n}{2}} {I}_{{x}^{′} , {y}^{′}}^{2} )}^{2}}}{\frac{1}{{( n + 1 )}^{2}} {\sum }_{x = i - \frac{n}{2}}^{i + \frac{n}{2}} {\sum }_{y = j - \frac{n}{2}}^{j + \frac{n}{2}} {I}_{x , y}^{2}} \qquad ( 2.2 )$$

where K is the speckle contrast value and I is the intensity of single detection unit from a 2D unit matrix detector. Equations 2.1 and 2.2 give speckle contrast value calculation, ratio between mean value and the standard deviation.

Devices and methods of the disclosure provide for the use of an optical detector that is sensitive to specific wavelengths in the visible and/or in the invisible range, for example, 400 nm to 1200 nm, aligned with the anatomical window surface, which enables data collection of the microcirculation in small blood vessels in a reliable way. The resolution of the detector is, for example, a range of 500×500 pixels to 1500×1500 pixels (where pixel is the smallest detector unit). The detector may be adapted for close-range measurements from the surface, for example, range of 1 mm to 15 mm, and detection of objects size width, for example, size range of 10 μm to 200 μm, such as capillary geometrical and physical parameters, for example, density, diameter and length. In addition, the detector resolution is designed to maintain speckle interference spatial size, for example, a range of 1 μm to 6 μm, e.g., as calculated according to equations 2.1 and/or 2.2. The detector may be a three-dimensional (3D; two spatial dimensions plus time dimension) sensor, which comprises a 2-dimensional array of small detectors that work synchronously in the time domain to achieve real-time measurement. The detector may be designed as a single unit or an array of two or more detectors, to gather reliable data independently or synchronically. The apparatus should collect and process data in physiological relevant time, for example, from ten samples per second to one sample in several minutes, to handle microcirculatory changes.

Desired speckle size is derived from the minimal detection object that generates the speckle—a blood cell. The blood cell mean diameter in the capillaries is 8 μm. To detect blood cell movement, speckle generated should be at least two times smaller. Thus, speckle size should be smaller than 4 μm.

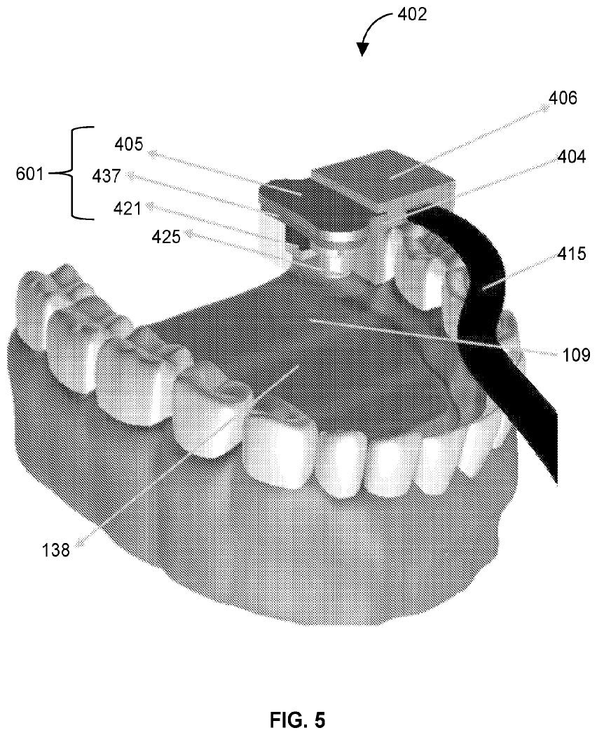

FIG. 6 shows optical elements 601 of a sensor 102 placed in an oral cavity 138 of a patient. As shown, the rigid support housing 105 carries a detector 121 for imaging the soft tissue 109 and the light source 125. The detector 121 and the light source 125 may be mounted to a board 603 (such as a printed circuit board) that is mounted in the housing 105. The board 603 may also include a processing unit 137 (e.g., such as an FPGA) communicatively coupled to the detector 121 and the light source 125.

The light source 125 may include optical element(s) 605, such as a lens or prism, for directing coherent light onto the soft tissue 109. Preferably, the light source 125 includes a LASER or a semiconductor optical amplifier as the source 125 and also further includes a focusing mechanism 609 as well as optionally a LASER tilting and expanding lens 613. The focusing mechanism 609 preferably includes one or more motors operable to position the light source 125 and the lens 613 with respect to an illuminated region 615 to provide focus for imaging operations. Any suitable motor may be used such as a linear motor or piezoelectric motor. For example, in some embodiments, the focusing mechanism is provided by a focus module package such as the focus module sold under the trademark M3-FS focus module by New Scale Technologies (Victor, N.Y.).

The detector 121 may include detection optical element(s) 606 for collecting the laser speckle image 201. Any suitable sensor may be used for the detector 121. In preferred embodiments, the detector 121 is provided by a CMOS camera sensor (certain embodiments use black and white—a monochrome sensor, or the sensor may be a color sensor). The optical elements 606 may be a main camera lens and the detector 121 may further include one or more optical filter(s) 633. The light source 121 illuminates an illuminated region 615 of the tissue 109 (preferably by coherent light such as created by a LASER device). The optical elements 606 may optionally include a light diffuser or, e.g., a beam homogenizer or array of microlenses to expand and shape the light beam. The detector captures an area 619 of the tissue. During an imaging operation, the focusing mechanism 609 may operate continually to ensure in-focus images. Focusing operations may be provided within the hardware of the focusing mechanism 609 (e.g., a focus module that includes a piezoelectric motor and control logic) or the focus mechanism 609 may include one or more motors to control positioning of the lens 613 under control of a autofocus module operating on the processing unit 137.

In preferred embodiments, a rigid, immobile dental anchor is attached to the teeth, then, the a housing of a sensor is attached to the dental anchor. The housing can be quickly attached and detached from the dental anchor and, by implication, the patient. The rigid attachment through the dental anchor provides for a repetitive measurement over the same area of tissue over time. The rigid dental anchor thus provides a spatial reference in the mouth cavity. The rigidity of the dental anchor with attached housing, plus the focusing module (e.g., with auto-focus operations implemented on a chip such as a field-programmable gate array) provide for meaningful, reproducible, and comparable imaging operations via laser speckle interference imaging over time, from the same region of tissue.

Sensor pixel resolution, for example, the number of a single unit matrix comprising the detector, is affected from two parameters: first, region of interest (ROI) of the sensor, and second, pixel size. The area of the detector 121, which comprises 2d matrix of the pixels area, will detect relevant tissue area. For example, 1 mm\hat{ }2 if tissue contains 50 capillaries.

For example, assuming 1× magnification is presented. 1 mm linear plan will contain approximately 10-25 capillaries. If mean capillary diameter is 50 μm, it will be detected at 50 μm area. With pixel size of 3 μm, 50 μm of capillary, will be detected with at least ˜15 pixels. To detect 25 capillaries, detected as 25×50=1.25 mm×1.25 mm detector will be used with above assumptions. The sensor 102 may include a system of narrow-band wavelength optic detectors 121 and optical filters (e.g., detection optical elements 606) that are designed to collect signal from wide range of wavelengths for each detector. The electro-optic system includes two or more detectors 121 that co-exist and independently collect data with high signal to noise ratio (SNR). Preferably, the rigid support structure 105 is configured to mount on maxillary or mandibular teeth or both 401 of the patient and emit the coherent light into palatal or sublingual capillaries within the tissue 109. In various embodiments, the Detector/Receiver may be Monochrome 2D array of pixel sensor unit with size of 1 um-5 um (such as a monochrome CMOS sensor).

The Detector/Receiver lens may include an optical objective that focuses the region of interest upon the detector.

Any Detector/Receiver optical filter (one or more) may include a selective light transmitter like polarizer, long pass filter and neutral density filter. The optical components may include a focusing mechanism discussed above.

The Transmitter may be Coherent laser (visible/nonvisible) source (monochromatic light) (Laser diode/VSCEL).

The Transmitter lens may include Hard/soft light lighting (Diffuser) to even the beam over region of interest.

A sensor of the disclosure may also separately include Non coherent illumination, e.g., such as a Visible light source (e.g., LED).

Devices and methods of the disclosure provide a sensor 102 that comprises a coherent energy source transmitter 125, the detector 121 and the optical unit that are aligned in a spatial position that allows the reflected speckle interference transmitted from the transmitter to be collected by the optic unit and be detected by the detector 121. A coherent energy transmitter 125 may be constructed with one or more small coherent energy transmitters with one or more wavelengths.

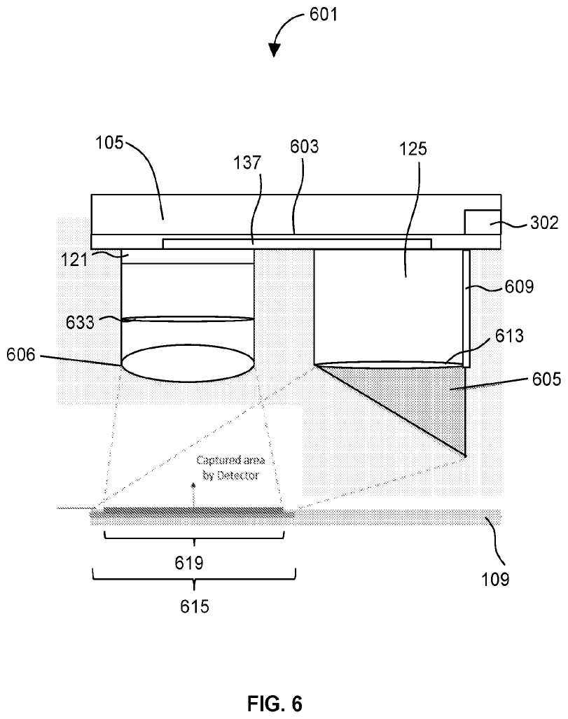

FIG. 7 is a block diagram of an electro-optical sensor unit system for use in devices and methods of the disclosure. The system preferably includes the sensor 102 and means 302 for transmitting a digital signal 305 from the processing unit 137 to the computer system 301. Any suitable hardware device(s) may be used for the means 302 for digital signal transmission. For example, the means 302 may include a wired connection (e.g., a flexible connection line 115) or wireless hardware such as a MIPI 2 USB controller. The sensor 102 is provided with a power supply such as a wired connection to power of a batter on the device.

The sensor 102 communicates with a computer system 301 communicatively coupled to the detector 121. The computer system 301 may be operable to: receive a digital signal from the light received from the speckle pattern by the detector and measure microcirculation in the tissue from the speckle pattern. Various tests have been performed demonstrating the feasibility of various aspects discussed herein.

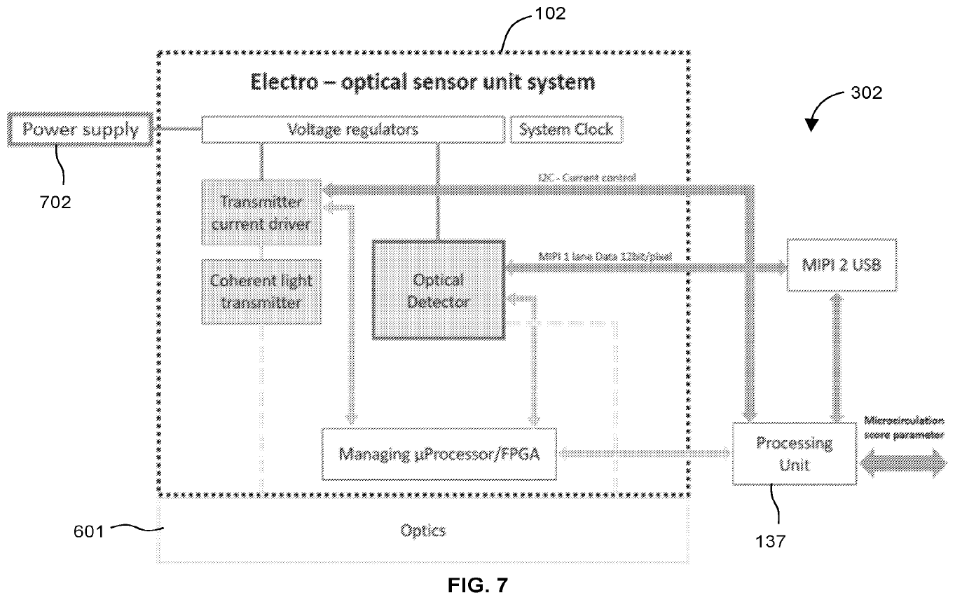

FIG. 8A shows raw speckle data taken by imaging a static system.

FIG. 8B shows speckle interference calculated from images of the system in a static state 801. Tests were performed in an ex vivo phantom system using a substrate having a micro channel through the substrate. The micro channel was filled with fluid and held stationary for imaging in a static state, and the fluid was flowed through the channel for imaging in dynamic state. Imaging the substrate shows the ability of the sensor 102 and detector 121 to show and detect conditions relevant to monitoring human microcirculation.

FIG. 8C shows speckle interference calculated from images of the system in a dynamic state 802.

The compute system 301 may be used to calculate a mean and a standard deviation from the speckle interference to provide a contrast related score. For example, for the system in the static state, the contrast related score is 72.3 (typically reported as a normalized score such as 0.723); for the system in the dynamic state, the contrast related score is 86.9 (e.g., normalized to 0.869). The visual distinction between the speckle interference calculated from images of the system in a dynamic state 802 and the speckle interference calculated from images of the system in a static state 801 is evident. Additionally, the computer is able to use contrast related scores to quantitatively and qualitatively evaluation, measure, show, report, and monitor microcirculation in a patient. The computer system 301 is operable to perform statistical calculation, e.g., mean and standard deviation ratio, to identify flow, e.g. as reported or shown by contrast related score. The speckle interference calculated from images of the system in a static state 801 shows measurement of a low flow rate.

The speckle interference calculated from images of the system in a dynamic state 802 shows measurement of a high flow rate by related contrast score performed on ex vivo phantom, for example, microfluidic chip with a channel's diameters of 100 μm, obtained using a laboratory system that was built according to systems described herein. The measurement performed on the ex vivo phantom showed that changes of flow rate in ex vivo phantom of small blood vessels may be observed with real-time laser speckle interference. As discussed, the computer system 301 may be used for measuring microcirculation by detecting fluctuation in the spatio-temporal domain of the speckle pattern.

FIG. 9 illustrates the spatio-temporal domain of the speckle pattern 901. Using the described information and devices, the computer system may provide to a physician information on blood flow in small blood vessels and a recommendation to decrease fluid delivery to avoid fluid overload. Features of the system include the rigid support structure 105, which may be configured for fitment to teeth 401 of the patient for hands-free positioning of the light source 125 and the detector 121 to image sublingual tissue 109 of the patient. Preferably the detector 121 and light source 125 are communicatively coupled to a computer station 301 operable for: continuous, hands-free monitoring, via the laser speckle pattern 201, of microcirculation while the patient is in shock, and to guide fluid delivery to treat shock or avoid hyper- or hypo-perfusion. The rigid support structure 105 may be configured for positioning within a mouth 138 of, and mounting on teeth 401 of, the patient to maintain the position of the detector 121 relative to soft, sublingual tissue 109 in the mouth while the detector 121 forms an image of the speckle pattern 201. The rigid support structure 105 preferably maintains position of the detector 121 relative to the soft, sublingual tissue 109 over time while the detector forms multiple co-registered images of speckle patterns 201, i.e., a spatio-temporal domain 901 of the speckle pattern 201 in the formed by coherent light from the source 125 in the sublingual tissue 109.

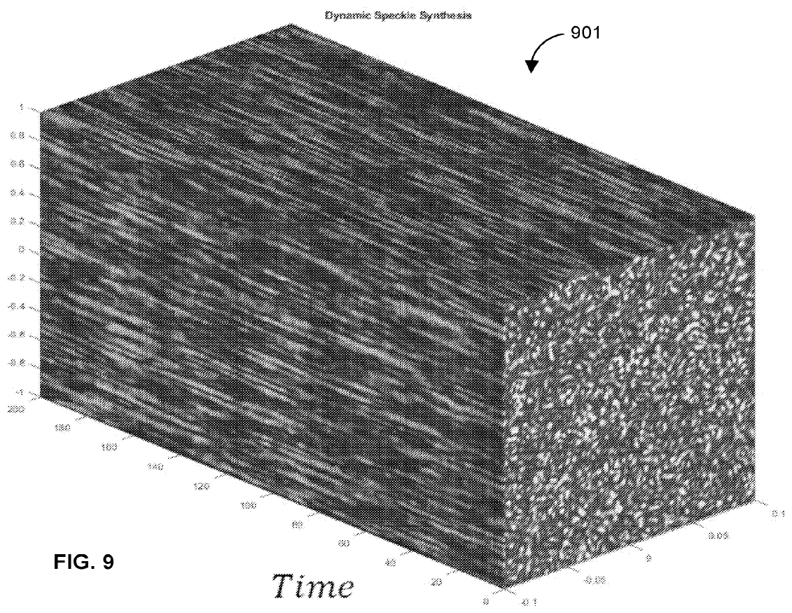

FIG. 10 diagrams a method 1001 of monitoring microcirculation. The method 1001 includes positioning 1003 a sensor 102 on tissue 109 of a patient. The sensor 102 includes an optical detector 121, a rigid structure 105 that holds the detector 102 in position relative to the tissue 109, and a light source 125 positioned on the rigid structure 105 to emit coherent light to form a speckle pattern 201 within the tissue 109. Preferably, the sensor includes an optical detector 121 and a rigid support housing 105 connected to a mounting tab 106 for attaching the detector 121 to an anchor 104. The anchor 104 attached to a patient and holds the detector 121 in position relative to tissue 109 of a patient. The sensor preferably includes a light source 125 with a lens 613 and a focusing mechanism 609. During operation, a motor of the focusing mechanism 609 may move the lens relative to the tissue 109. Other than lens motion of focusing (e.g., under control of auto focus logic implemented in circuitry within the sensor 102), parts of the sensor 102 such as the housing 105 and mounting tab 106 are held in a rigid position within the mouth of the patient during imaging without “hands-on” contact by a clinician. The rigid, immobile dental anchor attached to the teeth with the a housing of a sensor is attached provides for a repetitive measurement over the same area of tissue over time. The rigid dental anchor thus provides a spatial reference in the mouth cavity. The rigidity of the dental anchor with attached housing, plus the focusing module 609 (e.g., with auto-focus operations implemented on a chip such as an FPGA) provide for meaningful, reproducible, and comparable imaging operations via laser speckle interference imaging over time, from the same region of tissue. Because the method 1001 is for hands-free imaging (“set it and forget it”), the method 1001 includes leaving 1009 the sensor 102 positioned on the tissue 109 and performing hands-free imaging 1015 of the speckle pattern 201 with the detector 121. Further, the method preferably includes analyzing 1023 by a computer system images of the speckle pattern to monitor 1027 microcirculation in the patient. A processing unit on the sensor may form a digital signal comprising the images of the speckle pattern and transmits the digital signal to the computer system. The method 1001 may include positioning 1003 the sensor within an oral cavity 138 of the patient and leaving the sensor 1009 there for performing the hands-free imaging 1015. The positioning step may include mounting an anchor 104 of the sensor 102 on maxillary or mandibular teeth 401 of the patient and the imaging 1015 may include emitting the coherent light into palatal or sublingual capillaries, respectively, within the tissue, optionally while focusing the optical components 601 using a focusing mechanism 609. Positioning 1303 may include mounting the anchor 104 in a position that straddles mandibular teeth of the patient (see FIG. 4B) with the detector facing soft tissue in the oral cavity, such that a shape of the anchor grips the teeth with sufficient force to maintain the detector in position relative to the soft tissue.

The method 1001 is used for monitoring 1027. Monitoring involves more than one data point (image) obtained at different times, i.e., monitoring 1027 is a process that continues over time. Microcirculation in soft tissue is a dynamic process and laser speckle interference along requires more than one image. To do monitoring via laser speckle interference requires multiple images that are comparable to each other and thus that are taken of the same region of soft tissue. A sensor and system and apparatus of the disclosure provides for monitoring in way that cannot be performed with hand-held probes or even with optical probes that having moving parts (e.g., flexible tubes or hinges). A sensor 102 of the disclosure includes an anchor that grips the teeth 401, with a housing connected to the anchor, to maintain a position of the detector 121 and light source 125, with respect to soft tissue 109 over time to perform multiple hands-free imaging operations of one region of the soft tissue. The sensor is useful for hands-free, continuous monitoring. Preferably, the only moving parts include focusing mechanism operable to autofocus the lens on the tissue and any connecting hoses or wires (e.g., those flexible tubes or wires extending from the rigid structure and from the mouth of the patent for passing fluid, suction, power, or data to or from the sensor). An insight reflected in this disclosure is that using the disclosed hardware, a sensor may be mounted to a hard, skeletal part (e.g., teeth 401) of a patient for measuring microcirculation in soft tissue (e.g., sublingual, palatal, or buccal tissue). In preferred embodiments, the sensor 102 mounts fixedly to teeth 401 of the patient to image sublingual soft tissue by hands-free laser speckle interference imaging to provide continuous monitoring 1027 of microcirculation in the patient. The image is kept in focus despite motion of the tissue by virtue of a opto-electronic focusing module. Hand-held probes are not useful for monitoring 1027 by laser speckle interference because, like probes with moving parts, those probes do not have any mechanism for maintaining position over time of the imaging optics with respect to the tissue, and cannot make repetitive reproducible measurement of an area of tissue. Methods and systems of the disclosure provide for monitoring 1027 microcirculation by detecting fluctuation in the spatio-temporal domain of the speckle pattern 201. The method 1001 may include providing, by the computer system, a clinician with information on blood flow in small blood vessels and a recommendation to decrease fluid delivery to avoid fluid overload.

Methods 1001 and systems 101 of the disclosure provide for a rigid structure 105 configured for fitment to teeth 401 of the patient for hands-free positioning 1003 of the light source 125 and the receiver 121 to image 1015 sublingual tissue 109 of the patient and further wherein the receiver 121 and light source are communicatively coupled to a computer station 301 operable for: continuous, hands-free monitoring 1027, via the laser speckle pattern, microcirculation to guide fluid delivery to treat shock or avoid hyper- or hypo-perfusion. In such embodiments, the rigid structure grips teeth of the patient to hold the receiver and light source in position relative to tissue over time so that registration is maintained during formation of multiple laser speckle patterns in sublingual soft tissue. The method 1001 may also include providing by the computer system 301 to a clinician fluid delivery guidance to mitigate shock or avoid fluid overload.

Related aspects and embodiments provided by the disclosure include a general-purpose sensor for use in an oral cavity of a patient.

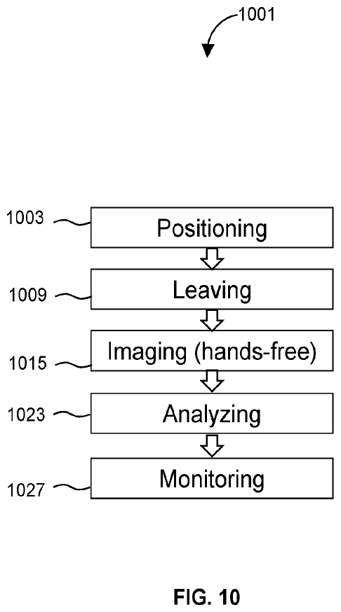

FIG. 11 shows a sensing device 1101 for use an oral cavity. The sensing device 1101 includes a support structure 1105 dimensioned to be placed and left within a mouth of a patient; and at least one sensor 1121 on a rigid portion 1106 of the structure.

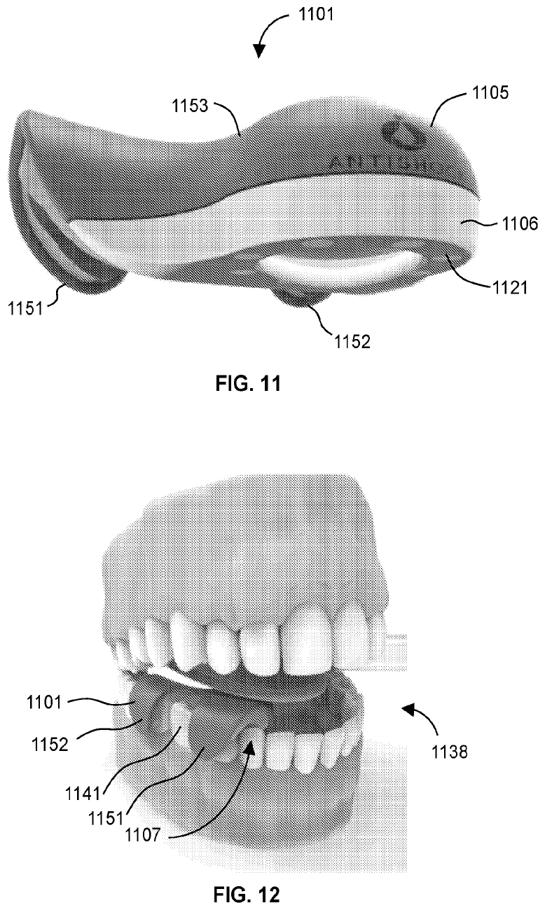

FIG. 12 shows the sensing device 1101 positioned within an oral cavity 1138. Part 1107 of the rigid portion has a fixed configuration complementary to tooth 1141 or bone of the patient whereby the rigid portion 1106 maintains the sensor 1121 in position to sense a vital function from a region of soft tissue in the mouth. As shown, the device 1101 includes a first hangar 1151 and a second hangar 1152 that are contoured to straddle the mandibular teeth 1141. The device 1101 may include a smooth, inert (e.g., plastic) cover 1153 to keep the tongue away from the sensor. The rigid portion 1106 maintains relative positioning between the sensor and the region of the soft tissue for multiple measurements over time. Once the structure if placed within the mouth, the sensor provides continuous and hands-free sensing of the vital function.

The sensing device 1101 may optionally include means for transmitting a signal to a computer system 301 (e.g., a wireless transceiver or a wired connection).

The sensing device 1101 provides a general purpose, in-mouth sensor for hands-free monitoring of patient vitals. The device 1101 may be deployed in an intensive care unit, an emergency vehicle, or out in the field for convenient and useful monitoring of patient vitals. The device 1101 may be used to sense and/or monitor any suitable vital function. For example, the device may be used to sense or monitor blood pressure, pulse, temperature, respiration, oxygen saturation, a blood sugar level, or a microcirculation parameter. For stable, hands-free placement, the device 1101 may have a fixed configuration configured to straddle a tooth of the patient. Preferably the fixed configuration is configured to straddle mandibular teeth of the patient with the sensor positioned to sense the vital function from sublingual tissue.

The following are parameters which the device may measure: Sublingual microcirculation parameters such as: microvascular flow index (MFI), DeBacker score, total vascular density (TVD), perfused vessel density (PVD), proportion of perfused vessels (PPV), vessels heterogeneity, red blood cell velocity, flow patterns; Temperature; Blood pressure; Total peripheral resistance; Heart rate and arrhythmia detection; Respiratory rate and respiratory pattern analysis; Oxygen saturation; Oxygen extraction; Depth of anesthesia; Blood components: such as Hemoglobin concentration, white cell count, and platelets count; Blood sugar levels; Blood analysis of chemistry components such electrolytes and clotting and coagulation and bleeding analysis; Humidity; Mechanical properties of blood such as blood viscosity; Red blood cell deformability; Blood vessel integrity; and Blood vessel calcification. Features may include that the sensor comprises a light detector; a transmitter on the rigid portion to emit coherent light into the region of tissue; a processing unit on the support structure that forms a digital signal of a speckle pattern sensed by the light detector, in any combination.

In certain embodiments, the device 1101 is used to monitor microcirculation. The remote computer system is operable to inform a clinician of one or more of impaired microcirculation, de-coherence between the microcirculation and macro-circulation, or a risk of fluid overload. The device 1101 may include a processing unit on the support structure to form a digital signal from a speckle pattern sensed by the light detector and send the digital signal to computer station, whereby the device and the computer station provide continuous, hands-free monitoring of microcirculation. Preferably, except for any connection line or wire extending from the device, and except for optional deformable foam in the hangars, the device 1101 provides a rigid positioning of sensing components with respect to tissue. The sensing components may include a transmitter on the rigid portion to emit coherent light into the region of tissue and may include focusing mechanisms to focus an imaging operation. The sensor may include a light detector operable to image a laser speckle pattern formed by the coherent light in the region of tissue, in which the device maintains the sensor in position over time and a focusing module focuses each imaging operation a region of tissue to form multiple, co-registered laser speckle images of the region of tissue.

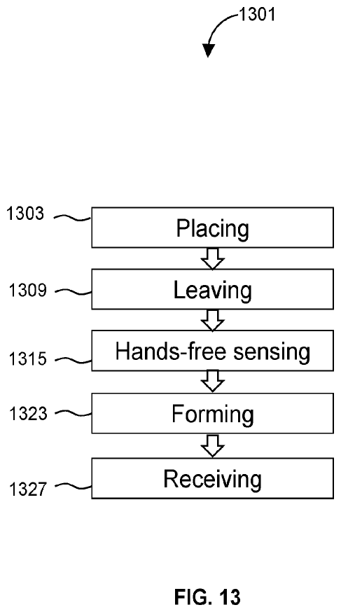

FIG. 13 shows steps of a method 1301 for sensing a vital function. The method 1301 comprising: placing 1303 and leaving 1309 a sensing device 1101 in a mouth 1138 of a patient. The device 1101 includes a housing 1105 carrying a sensor 1121 on a rigid portion 1106 of the structure. Part of the rigid portion has a fixed configuration complementary to tooth 1141 or bone of the patient whereby the rigid portion maintains the sensor in position to sense a vital function from a region of soft tissue in the mouth. The method 1301 includes operating the device for hands-free sensing 1315 of the vital function. The method 1301 preferably includes forming 1323, by a processing unit on the support structure, a digital signal representing the vital function, and receiving 1327 the digital signal at a computer system the provides a clinician with information about the vital function. The sensor 1101 provides continuous and hands-free sensing of the vital function. The method 1301 includes a “leaving” step 1309, showing that the sensor is placed in the patient and the clinician walks away—leaving the sensor 1101 to perform hands-free, continuous, non-invasive sensing. Preferably the vital function is one selected from the group consisting of blood pressure, pulse, temperature, respiration, oxygen saturation, a blood sugar level, and a microcirculation parameter or Sublingual microcirculation parameters such as: microvascular flow index (MFI), DeBacker score, total vascular density (TVD), perfused vessel density (PVD), proportion of perfused vessels (PPV), vessels heterogeneity, red blood cell velocity, flow patterns; Temperature; Blood pressure; Total peripheral resistance; Heart rate and arrhythmia detection; Respiratory rate and respiratory pattern analysis; Oxygen saturation; Oxygen extraction; Depth of anesthesia; Blood components: such as Hemoglobin concentration, white cell count, and platelets count; Blood sugar levels; Blood analysis of chemistry components such electrolytes and clotting and coagulation and bleeding analysis; Humidity; Mechanical properties of blood such as blood viscosity; Red blood cell deformability; Blood vessel integrity; and Blood vessel calcification. The rigid portion maintains relative positioning between the sensor and the region of the soft tissue for multiple measurements over time. Once the structure is placed within the mouth, the sensor provides continuous and hands-free sensing of the vital function. Preferably the fixed configuration is configured to straddle a tooth of the patient. The method 1301 may include emitting coherent light into the sublingual tissue by an optical transmitter on the rigid portion. The method 1301 may include forming a digital image of a speckle pattern detected in the tissue by the sensor via a processor on the sensing device.

For additional background see U.S. Pat. No. 10,070,796 B2; U.S. Pat. No. 9,226,661 B2; and WO 2017/083587 A1, all incorporated by reference.

Systems and methods of the disclosure provide sensor to be placed in the mouth cavity environment, which possesses a miniaturized, optically aligned and mechanically stable design. With a combination of components of the shelf (CMOS camera, laser diode, LED, lens, diffuser and optical filter) and mechanical fixation, the sensor may have dimensions of, e.g., about 7 mm×14 mm×9 mm (width×length×height). The disclosure provides a low-cost, compact sensor that will acquire reliable data and extract microcirculation (blood flow dynamics) parameters, stable and continuous measurement of repeatable sublingual capillaries that reflects well the systemic micro-hemodynamics, for example, MFI (Mean Flow Index), DeBacker score, etc. In order to be able to extract microcirculation parameters, the sensor is be able to detect clinically significant changes in blood velocity.

With preclinical trials mechanical set-up, real-time laser speckle interference methods were used to measure the capillaries with a minimum diameter of 30 micrometer.

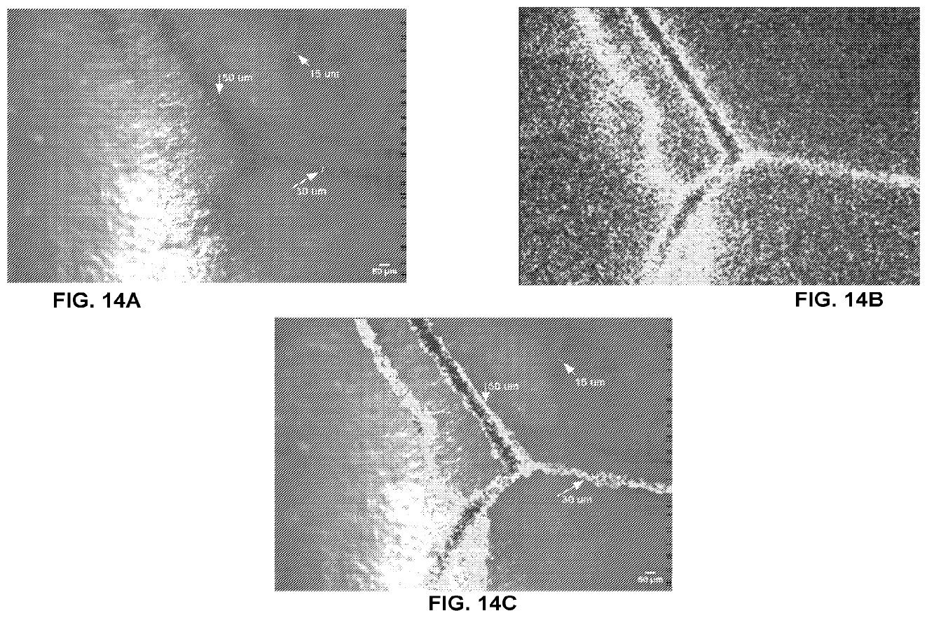

FIG. 14A is an image taken with visible (green) light illumination in preclinical trials.

FIG. 14B is an image after laser speckle contrast analysis in the preclinical trials.

FIG. 14C is a combined and overlapped dynamic flow over white field image from the preclinical trials.

Systems and methods of the disclosure provide a mechanical system that will support the sensor's electro-optical system in the oral cavity environment, such as the sublingual area, and enable stability and accuracy in spatial (up to drift of 2-5 [um]) and temporal domains for real-time and continuous measurement for continuous measurement.

Systems and methods of the disclosure provide for the implementation of the speckle phenomenon in the dynamic biological environment for a prolonged period, for example, between 4-6 hrs., and acquire reliable data repetitively from a selected area in real-time and continuous mode in a operator independent fashion. The blood flow measurement should be at least 0.8 [mm/sec] to provide reliable data for further analysis by a unique algorithm.

Systems and methods of the disclosure provide a system useful to inform a clinician of one or more of impaired microcirculation, de-coherence between the microcirculation and microcirculation, or a risk of fluid overload.

Systems and methods of the disclosure provide a non-invasive sublingual apparatus in a system for calculating fluid administration instructions for treating impaired tissue perfusion in a patient, which comprises: a support structure capable of supporting sublingual placement in a patient's oral cavity; and an array of multiple electro-optical transceivers mechanically connected to the support structure which emit coherent wave to form a speckle pattern on a sublingual target area in a base of the oral cavity and to capture a reflection of the formed speckle pattern from the sublingual target area.

The transceivers may include a plurality of light-directing lenses and optics.

Systems and methods of the disclosure may include software capable of synchronically instructing the array of multiple electro-optical transceivers to form the speckle pattern, processing the reflection to reconstruct spatiotemporal data mapping perfusion in a plurality of sublingual capillaries located at the oral cavity base over time, performing an analysis of the spatiotemporal data to identify changes in the perfusion in at least some of the plurality of sublingual capillaries, and calculating fluid administration instructions for treating impaired tissue perfusion of the patient according to the analysis and manage IV fluids administration

Systems and methods of the disclosure may be used for repetitive measuring of blood flow in multiple capillaries over time from a selected area.

Systems and methods of the disclosure may be used in a method of adjusting fluid administration to treat impaired tissue in a patient, which comprises: applying an apparatus such as sensor 102 to the patient; collecting data generated by the apparatus; analyzing the data; and providing instructions to an operator to adjust fluid administration.

Systems and methods of the disclosure may include technology, elements, or features discussed in any of US 2004/0006263; US 2009/0269716; WO 2015/107109; and/or US 2016/0220129, the entire contents of each of which are incorporated by reference for all purposes.

Aspects provide a system 101 for monitoring microhemodynamics. The system 101 includes an anchor 104 that can be fastened to one or more teeth 401 of a patient; a sensor housing 105 detachably connectable to the anchor; and a laser speckle interference imaging subsystem 601 carried by the sensor housing 105, wherein when the anchor 104 is anchored to the tooth 401 of the patient, and when the sensor housing 105 is attached to the anchor 104, the laser speckle interference imaging subsystem 601 is held in position with respect to the teeth. The anchor may include a clamp 139 for fastening the anchor to one or more teeth of the patient. Preferably the sensor housing 105 includes a quick-release attachment mechanism 161 that places the laser speckle interference imaging subsystem 601 back into the position with respect to the tooth or bone when the sensor housing 105 is detached and re-attached to the anchor 104. The laser speckle interference imaging subsystem 601 may include a coherent light source 125 that emits light towards soft tissue 109 when the sensor housing 105 is attached to the anchor 104, fastened to the teeth, and a focusing module that refocuses the light as the tissue moves with respect to the tooth or bone. The focusing module may include a lens 613 through which the light passes, motor 609 for positioning the lens, and focus-control logic implemented in a processor 137 within the sensor housing 105. The laser speckle interference imaging subsystem 601 may include an optical detector 121, such as a CMOS or sCMOS sensor for detecting a laser speckle interference pattern in the soft tissue, and optionally an array of optical detectors 121. The system may include a processing unit 137 in the sensor housing 105 communicably coupled to the laser speckle interference imaging subsystem 601, and a remote computer system 301 communicably coupled to the processing unit 137. The computer system 301 may be included to provide a display or report showing parameters of microcirculation in the patient to a clinician and optionally provides guidance for changing a level of fluid delivery. Preferably once the sensor housing 105 is connected to the anchor 104, with the anchor 104 fastened to the tooth 401 of the patient, the sensor housing 105 and anchor 104 fit entirely within an oral cavity 138 and provide hands-free measurement of microcirculation in sublingual soft tissue 109. In some embodiments, the sensor housing 105 includes an attachment mechanism that places the laser speckle interference imaging subsystem back into the position with respect to the tooth or bone when the sensor housing is detached and re-attached to the anchor for reproducible positioning and wherein the laser speckle interference imaging subsystem includes a coherent light source emits light towards soft tissue when the sensor is attached to the anchor, anchored to the tooth or bone, and a focusing module that refocuses the light as the tissue moves with respect to the tooth or bone for consistent focusing, wherein the reproducible positioning and consistent focusing provide for repeatable and comparable measurements over time.

1.-15. (canceled)

16. A method of monitoring microcirculation, the method comprising:
positioning a sensor on tissue of a patient, wherein the sensor comprises an optical detector, a rigid housing that holds the detector in position relative to the tissue, and a light source positioned on the rigid housing to emit coherent light to form a speckle pattern within the tissue;
leaving the sensor positioned on the tissue and performing hands-free imaging of the speckle pattern with the detector; and
analyzing by a computer system images of the speckle pattern to monitor microcirculation in the patient.

17. The method of claim 16, wherein the positioning step includes attaching the sensor to an anchor anchored to teeth of the patient.

18. The method of claim 16, wherein the hands-free imaging includes multiple imaging operations over time, wherein each imaging operation includes moving a lens by a focusing module in the sensor to provide focus for that imaging operation.

19. The method of claim 16, wherein a processing unit on the sensor forms a digital signal comprising the images of the speckle pattern and transmits the digital signal to the computer system, wherein the computer system is operable to reconstruct spatiotemporal data mapping perfusion in a plurality of capillaries located in the tissue.

20. The method of claim 16, further comprising attaching the sensor via a quick-release attachment mechanism to an anchor within an oral cavity of the patient and leaving the sensor within the oral cavity for performing the hands-free imaging.

21. The method of claim 20, wherein the anchor is mounted on teeth of the patient and the imaging includes emitting the coherent light through a focusing module on the sensor into palatal or sublingual capillaries, respectively, within the tissue.

22. The method of claim 20, wherein the anchor is mounted in a position that straddles mandibular teeth of the patient with the detector facing soft tissue in the oral cavity, wherein a mechanism of the anchor grips the teeth with sufficient force to maintain the detector in position relative to the soft tissue.

23. The method of claim 22, wherein the anchor maintains a position of the detector over time, and the focusing module repeated focuses the light source, to perform multiple hands-free imaging operations of one region of the soft tissue.

24. The method of claim 22, wherein, when anchored to the teeth via the anchor, the sensor comprises no flexible or moveable part other than the focusing module and any one or more flexible tubes or wires extending from the rigid structure and from the mouth of the patent, the tubes or wires passing fluid, suction, power, or data to or from the sensor.

25. The method of claim 16, wherein the sensor mounts fixedly to teeth of the patient to image sublingual soft tissue by hands-free laser speckle interference imaging to provide continuous monitoring of microcirculation in the patient.

26.-27. (canceled)

28. The method of claim 16, wherein the sensor is configured for attachment to an anchor attached to teeth of the patient for hands-free positioning of the light source and the receiver to image sublingual tissue of the patient and further wherein the receiver and light source are communicatively coupled to a computer station operable for: continuous, hands-free monitoring, via the laser speckle pattern, microcirculation while the patient is in shock, and to guide fluid delivery to treat shock or avoid fluid overload.

29. The method of claim 28, wherein the anchor grips teeth of the patient to hold the receiver and light source in position relative to tissue over time so that registration is maintained during formation of multiple laser speckle patterns in sublingual soft tissue.

30.-56. (canceled)

57. A system for monitoring microhemodynamics, the system comprising:
an anchor fastenable to tooth or bone of a patient;
a sensor housing detachably connectable to the anchor; and
laser speckle interference imaging subsystem carried by the sensor housing, wherein when the anchor is anchored to the tooth or bone of the patient, and when the sensor housing is attached to the anchor, the laser speckle interference imaging subsystem is held in position with respect to the tooth or bone.

58. (canceled)

59. The system of claim 57, wherein the sensor housing includes a quick-release attachment mechanism that places the laser speckle interference imaging subsystem back into the position with respect to the tooth or bone when the sensor housing is detached and re-attached to the anchor.

60. The system of claim 57, wherein the laser speckle interference imaging subsystem includes a coherent light source emits light towards soft tissue when the sensor is attached to the anchor, anchored to the tooth or bone, and a focusing module that refocuses the light as the tissue moves with respect to the tooth or bone.

61. The system of claim 60, wherein the focusing module includes a lens through which the light passes, motor for positioning the lens, and focus-control logic implemented in a processor within the sensor housing.

62. The system of claim 57, wherein the laser speckle interference imaging subsystem includes an optical detector for detecting a laser speckle interference pattern in the soft tissue.

63.-64. (canceled)

65. The system of claim 57, wherein once the sensor housing is connected to the anchor, the anchor fastened to the tooth of the patient, the sensor housing and anchor fit entirely within an oral cavity and provide hands-free measurement of microcirculation in sublingual soft tissue.

66. The system of claim 57, wherein the sensor housing includes an attachment mechanism that places the laser speckle interference imaging subsystem back into the position with respect to the tooth or bone when the sensor housing is detached and re-attached to the anchor for reproducible positioning and wherein the laser speckle interference imaging subsystem includes a coherent light source emits light towards soft tissue when the sensor is attached to the anchor, anchored to the tooth or bone, and a focusing module that refocuses the light as the tissue moves with respect to the tooth or bone for consistent focusing, wherein the reproducible positioning and consistent focusing provide for repeatable and comparable measurements over time.

67. The system of claim 57, wherein the laser speckle interference imaging subsystem includes an array of optical detectors for detecting a laser speckle interference pattern in the soft tissue.
